(19) United States
(12) Patent Application Publication
(10) Pub. No.: US 2006/0147519 A1
(43) Pub. Date: Jul. 6, 2006

(54) Process for the preparation of robust formulations of valacyclovir hydrochloride tablets

(76) Inventors: Ashish Gogia, NEW DELHI, DELHI (IN); Romi Barat Singh, Uttar Pradesh (IN); Pananchukunnath Manoj Kumar, Delhi (IN); Sunilendu Bhushan Roy, Maharashtra (IN); Rajiv Malik, Wein (AT)

Correspondence Address: RANBAXY INC., 600 COLLEGE ROAD EAST, SUITE 2100, PRINCETON, NJ, 08540, US

(21) Appl. No.: 10/518,972

(22) PCT Filed: Jun. 24, 2003
(86) PCT No.: PCT/IB03/02456

§ 371 Date: Jan. 18, 2006

(30) Foreign Application Priority Data
Jun. 24, 2002 (IN) 673/DEL/2002

(51) Int. Cl. A61K 9/20; A61K 31/522

(52) U.S. Cl. 424/464; 514/263.38

(57) ABSTRACT

The invention relates to robust formulations of valacyclovir hydrochloride tablets. A robust tablet includes a hydrated form of valacyclovir hydrochloride having a water of hydration content of more than approximately 3% w/w and a particle size of less than approximately 355 μm.

### FIELD OF THE INVENTION

The technical field of the invention relates to robust formulations of valacyclovir hydrochloride tablets.

### BACKGROUND OF THE INVENTION

Valacyclovir is the L-valine ester of acyclovir and has been shown to possess anti-viral properties. The hydrochloride salt is the preferred form of this compound. Valacyclovir and its salts, including the hydrochloride salt are disclosed in U.S. Pat. No. 4,957,924 (e.g., Example 1B), European Patent No. 0308065 (e.g., Example 1B), and Beauchamp et al., Antiviral Chemistry and Chemotherapy, 3(3), 157-164 (1992) (e.g., page 162, column 1). Tablets of valacyclovir are also generally disclosed in the U.S. Pat. No. 4,957,924 and European Patent No. 0308065.

In U.S. Pat. No. 6,107,302, Carter et al. have disclosed that valacyclovir hydrochloride can exist in various forms, particular among them is an anhydrous and crystalline valacyclovir hydrochloride. This anhydrous form may have a water of hydration content up to 3% by weight. This anhydrous crystalline valacyclovir hydrochloride has been shown to be chemically and physically stable with good formulation and storage properties.

In U.S. Pat. No. 5,879,706, Carter et al. also have disclosed that during the development of a tablet formulation containing a high proportion of the drug they often encountered difficulty in obtaining tablets of sufficient hardness and friability necessary for handling and for film coating. As per the U.S. Pharmacopoeial requirements, tablets should have friability not exceeding 1%. If the tablets are too friable, they will chip or break during coating, packaging and transport.

In an effort to increase the hardness of the tablets and improve their friability, the inventors of U.S. Pat. No. 5,879,706, tried several remedies such as increasing the compression force, decreasing the amount of lubricant, and increasing binder concentration without any success. Eventually they found a tablet formulation containing 0.05% to 3% w/w colloidal silicon dioxide and extragranular microcrystalline cellulose as the filler, and which was robust with substantially improved friability and hardness. Apparently, colloidal silicon dioxide and extragranular microcrystalline cellulose appear to have a synergistic effect such that robust tablets of valacyclovir can consistently be made to an acceptable hardness without introducing stress cracks during high compression force (e.g., tableting process). This formulation also had satisfactory disintegration time and lubrication properties. An anhydrous crystalline form of valacyclovir hydrochloride having not more than 3% water of hydration was used as the active ingredient in the '706 patent.

### SUMMARY OF THE INVENTION

In one general aspect, there is provided a tablet that includes a hydrated form of valacyclovir hydrochloride having a water of hydration content of more than approximately 3% w/w and a particle size of less than approximately 355 μm.

Embodiments of the tablet may include one or more of the following features. For example, the valacyclovir hydrochloride may have a water of hydration content of more than approximately 4% w/w. The valacyclovir hydrochloride may have a water of hydration content of between approximately 3% w/w and approximately 16% w/w. The valacyclovir hydrochloride may have a particle size of less than approximately 250 μm. The valacyclovir hydrochloride concentration may be at least approximately 50% w/w of the tablet. The tablet may have a friability and the friability of the tablet does not exceed approximately 1% w/w. The tablet may have a hardness and the hardness of the tablet is at least approximately 10 kP.

The tablet may further include one or more pharmaceutically acceptable excipients. The pharmaceutically acceptable excipients may be one or more of a filler, binding agent, disintegrant, and lubricant. The filler may be one or more of dicalcium phosphate and microcrystalline cellulose. The filler may be from about 5% to about 40% w/w of the tablet. The binding agent may be one or more of hydroxypropyl methylcellulose, hydroxypropyl cellulose, and polyvinyl pyrrolidone. The binding agent may be between 0.05% and 5% w/w of the tablet. A portion of the binding agent may be present extra granularly as a dry binding agent and the extra granular dry binding agent may be between approximately 0.05% and approximately 2% w/w of the tablet. The disintegrant may be one or more of clays, kaolin, bentonite, veegum; celluloses, microcrystalline cellulose, croscarmellose sodium, non-ionic disintegrants, and crospovidone and the disintegrant may be from approximately 0.5% to approximately 7% w/w of the tablet. The tablet may further include a film coating. The tablet may be free or substantially free of both silicon dioxide and microcrystalline cellulose.

In another general aspect, a tablet includes an intragranular portion and an extragranular portion. The intragranular portion includes at least approximately 50% w/w of a hydrated form of valacyclovir hydrochloride having a water of hydration content of more than approximately 3% w/w and a particle size less than approximately 355 μm, at least one filler, at least one binding agent, and at least one disintegrant. The extragranular portion includes at least one lubricant. The friability of the tablet does not exceed approximately 1% and the hardness is at least approximately 10 kP.

In another general aspect, a tablet includes an intragranular portion and an extragranular portion. The intragranular portion includes at least approximately 50% w/w of a hydrated form of valacyclovir hydrochloride having a water of hydration content of more than approximately 3% w/w and particle size less than approximately 355 μm, at least one filler, at least one binding agent, and at least one disintegrant present within the granules of the tablet. The extragranular portion includes at least one lubricant and at least one binding agent. The friability of the tablet does not exceed approximately 1%, the hardness is at least approximately 10 kP.

Embodiments of the tablet may include one or more of the following features. For example, the binding agent in the intragranular portion and the binding agent in the extragranular portion are of the same material composition.

In another general aspect, a method of treatment of a viral infection in a mammal comprising administering to the mammal one or more tablets to administer an effective anti-viral amount of valacyclovir hydrochloride, the tablet comprising a hydrated form of valacyclovir hydrochloride having a water of hydration content of more than approximately 3% w/w and a particle size of less than approximately 355 μm.

Embodiments of the method of treatment may include one or more of the following features. For example, the virus may be a DNA virus. The virus may be one or more of herpes simplex 1, herpes simplex 2, varicella zoster, cytomegalovirus, Epstein-Barr viruses, human herpes virus-6 HIV-6), and hepatitis B virus. The virus may be one or more of a papilloma or wart virus. The method may further include administering the tablet with a second active compound. The second active compound may include zidovudine.

In another general aspect, there is provided a process for preparing a tablet that includes at least approximately 50% w/w of a hydrated form of valacyclovir hydrochloride having a water of hydration content of more than approximately 3% w/w and a particle size less than approximately 355 μm and one or more of at least one binding agent, at least one filler, at least one disintegrant and at least one lubricant. The process includes forming granules of valacyclovir hydrochloride; and blending an optional portion of the at least one binding agent and a lubricant with the granules. The hardness of the tablet is at least approximately 10 kP and the friability is not more than approximately 1%.

Embodiments of the process may include one or more of the following features. For example, forming the granules may include mixing the valacyclovir hydrochloride and the one or more of the at least one binding agent, the at least one filler, and the at least one disintegrant; granulating with a granulating solution to form granules; drying the granules; blending the granules with a lubricant; and compressing the blended mixture to form a tablet. Blending the granules with a lubricant may further include blending with a binding agent.

Forming the granules may include dissolving the binding agent in a granulating solution; adding and mixing to the granulating solution the valacyclovir hydrochloride and the one or more of the at least one binding agent, the at least one filler, and the at least one disintegrant; granulating with a granulating solution to form granules; drying the granules; blending the granules with a lubricant; and compressing the blended mixture to form a tablet. Blending the granules with a lubricant further may include blending with a binding agent.

The granulation may results in a fluid uptake of between 8-16%. The fluid uptake after granulation may between approximately 12% and approximately 16%. The granules may be dried to a moisture content of more than approximately 4% w/w. The extra granular binding agent may be first blended with the lubricant before blending with the granules. The extra granular binding agent may be added separately from the lubricant.

In another general aspect, a method of improving one or both of friability and hardness of a tablet that includes valacyclovir hydrochloride includes reducing the particle size of a hydrated form of valacyclovir hydrochloride. The valacyclovir hydrochloride has a water of hydration content of more than approximately 3% w/w.

Embodiments of the method may include one or more of the features described above including, for example, the particle size being less than approximately 355 μm.

In another general aspect, a method of improving one or both of friability and hardness of a tablet that includes valacyclovir hydrochloride having a particle size of less than approximately 355 μm. The method includes forming the tablet from a hydrated form of valacyclovir hydrochloride having a water of hydration content of more than approximately 3% w/w.

In another general aspect, there is provided a tablet having a hydrated form of valacyclovir hydrochloride and being characterized by the absence of colloidal silicon dioxide and extra granular microcrystalline cellulose. Embodiments of the tablet may include one or more of the features described above. For example, the valacyclovir hydrochloride may have a water of hydration content of more than approximately 3% w/w and a particle size of less than approximately 355 μm.

The details of one or more embodiments of the inventions are set forth in the description below. Other features, objects, and advantages of the invention will be apparent from the description and claims.

### DETAILED DESCRIPTION OF THE INVENTION

The inventors have found that using a hydrated form of valacyclovir hydrochloride having a particle size of less than 355 μm in a tablet formulation provides an effective method of overcoming the above friability and hardness problems without the need to use the combination of colloidal silicon dioxide and extra granular microcrystalline cellulose. The inventors also have found that the hardness can be further improved by increasing the moisture content of granules and/or using extra granular binding agent in the tablet formulation.

The term “hydrated form of valacyclovir hydrochloride” as used herein means valacyclovir hydrochloride having a water of hydration content of more than 3% by weight of the valacyclovir hydrochloride, wherein the water of hydration refers to a combination of water with valacyclovir hydrochloride and the water retains its molecular state and is either absorbed, adsorbed or contained within a crystal lattice of the substrate molecules of Valacyclovir hydrochloride. The term “adsorbed” as used herein refers to the physical state in which the water molecule in the hydrated valacyclovir hydrochloride is distributed over the surface of the solid hydrated valacyclovir hydrochloride. The term “absorbed” as used herein refers to the physical state in which the water molecule in the hydrated valacyclovir hydrochloride is distributed throughout the body of the solid hydrated valacyclovir hydrochloride.

Hydrated valacyclovir hydrochloride may contain water of hydration content of approximately 3 to 16% by weight, and more particularly approximately 3.1 to 15% by weight. The water of hydration of hydrated forms of valacyclovir hydrochloride in any batch of the compound can be measured by the overall water of hydration content of each batch. This water of hydration content is measured by the Karl Fischer method, which is well known in the art and is described in the 1990 U.S. Pharmacopoeia at pages 1619-1621, and the European Pharmacopoeia, second edition (1992), part 2, sixteenth fascicule at v. 3.5.6-1.

Therefore, in one general aspect there is provided a stable and robust tablet formulation that includes a hydrated form of valacyclovir hydrochloride having a water of hydration content of more than approximately 3% w/w and a particle size of less than approximately 355 μm. In another general aspect, there is provided a stable and robust tablet formulation that includes a hydrated form of valacyclovir hydrochloride having a water of hydration content of more than approximately 4% w/w and a particle size of less than approximately 250 μm. In another aspect, there is provided a tablet formulation that includes at least approximately 50% w/w hydrated form of valacyclovir hydrochloride having water of hydration content of more than approximately 3% w/w and particle size less than approximately 355 μm, a filler, a binding agent, a disintegrant present within the granules of the tablet and a lubricant being present extra granularly; wherein the friability of the tablet does not exceed approximately 1% and the hardness is at least approximately 10 kP.

In another aspect, there is provided a tablet formulation comprising at least about 50% w/w hydrated form of valacyclovir hydrochloride having water of hydration content of more than approximately 3% w/w and particle size less than approximately 355 μm, a filler, a binding agent, a disintegrant present within the granules of the tablet, a lubricant and at least a portion of the binding agent being present extra granularly; wherein the friability of the tablet does not exceed approximately 1% and the hardness is at least approximately 10 kP. In another general aspect, there is provided a robust tablet formulation of valacyclovir hydrochloride having a friability not exceeding approximately 1% and a hardness of at least approximately 10 kP and the tablet is capable of being film coated.

A further aspect provides a process for preparing a tablet formulation includes at least about 50% w/w hydrated form of valacyclovir hydrochloride having water of hydration content of more than approximately 3% w/w and particle size less than approximately 355 μm, a binding agent, a filler, a disintegrant, and a lubricant, wherein the hardness of the tablet is at least approximately 10 kP, the friability is not more than approximately 1%. The process includes the steps of forming granules that contain the valacyclovir hydrochloride and blending the lubricant with said granules.

A further aspect provides a process for preparing a tablet formulation that includes forming granules by mixing the valacyclovir hydrochloride, optionally a binding agent or a portion thereof, optionally a portion of a filler and optionally a portion of a disintegrant, granulating with a granulating solution to form granules or dissolving the binding agent or a portion in the granulating solution before adding to valacyclovir; drying the granules; blending the granules with optionally a portion of a binding agent and a lubricant and then compressing the blended mixture to form a tablet.

A further aspect provides a process for preparing a tablet formulation that includes forming granules by mixing the valacyclovir hydrochloride, optionally a binding agent or a portion thereof, optionally a portion of a filler and optionally a portion of a disintegrant, granulating with a granulating solution to form granules or dissolving the binding agent or a portion in the granulating solution before adding to valacyclovir, so that the fluid uptake is not more than 18% w/w, drying the granules; blending the granules with optionally a portion of a binding agent and a lubricant and then compressing the blended mixture to form a tablet.

In another aspect, there is provided a process for preparing a tablet that contains at least approximately 50% w/w hydrated form of valacyclovir hydrochloride having a water of hydration content more than approximately 3% w/w and particle size less than approximately 355 μm, a binding agent, a filler, a disintegrant and a lubricant wherein the hardness of the tablet is at least 10 kP, the friability is not more than 1%; said process comprising the steps of forming granules by mixing the valacyclovir or its salt, optional binding agent or a portion thereof, optionally a portion of filler; and optionally a portion of a disintegrant; granulating with a granulating fluid to form granules or dissolving the binding agent or a portion thereof in the granulating fluid before adding to valacyclovir; drying the granules to a moisture content of more than approximately 4% w/w; blending the granules with at least a portion of the binding agent and a lubricant; and then compressing the blended mixture to form a tablet.

In another aspect, there is provided a tablet for use in medical therapy, e.g. in the treatment of diseases caused by various DNA viruses, such as herpes infections, for example, herpes simplex 1 and 2, varicella zoster, cytomegalovirus, Epstein-Barr viruses or human herpes virus-6 (HIV-6) as well as diseases caused by hepatitis B. The tablet may also be used for the treatment of papilloma or wart virus infections and, may furthermore be administered in combination with other therapeutic agents, for example with zidovudine, to treat retroviral associated infections in particular HIV infections.

In addition to its use in human medical therapy, the tablet can be administered to other animals for treatment of viral diseases, e.g., to other mammals. The present invention also provides a method for the treatment of a viral infection, particularly a herpes viral infection, in an animal, e.g. a mammal such as a human, which comprises administering to the host one or more of said tablet to provide an effective antiviral amount of the active compound.

Valacyclovir is a high dose drug and the drug typically makes up at least 50% of the weight of a tablet that includes valacyclovir. The drug characteristics therefore play an important role in determining the characteristics of the final formulation When formulating tablets with anhydrous crystalline valacyclovir hydrochloride, the inventors encountered problems similar to those described in U.S. Pat. No. 5,879,706. However, when using a hydrated form of valacyclovir having a water of hydration content more than 3% w/w and a drug particle size less than 355 μm, the inventors surprisingly found that the problems of friability and low hardness, etc disappeared. Without intending to be limited by any theory, the inventors believe that the increased robustness of the tablets containing high amounts of the hydrated drug may result from the water of hydration helping in the binding of the drug and excipients and thereby result in a granulate that has better compressibility characteristics.

The particle size of the drug also played an important role in providing the desired compressibility, hardness and friability of the tablet formulation. The particle size of the drug was maintained at less than 355 μm, and in particular less than 250 μm.

A tablet formulation in accordance with the present invention further contains one or more pharmaceutically acceptable excipients such as those belonging to the category of fillers, binding agent, disintegrants, lubricants and the like.

The fillers may be any suitable pharmaceutically acceptable filler, including one or more of calcium hydrogen phosphate, lactose, microcrystalline cellulose and the like.

The binding agent may be any suitable binding agent commonly known in the art, including one or more of cellulose ethers (e.g., hydroxypropyl methylcellulose and hydroxypropyl cellulose) and polyvinyl pyrrolidone, such as the pyrrolidone that is sold under the trade name povidone and is available as K30, and more preferably K90. The binding agent is present at from about 0.5% to about 5% w/w of the tablet. The inventors also have observed that the use of cellulose ethers as an extragranular dry binding agent increases the hardness and reduces the friability of the tablet. The extragranular dry binding agent is present at from about 1% w/w to about 5% w/w of the tablet; in particular it is present at about 1% w/w to about 2% w/w of the tablet.

The disintegrants may be present at about 0.5% w/w to about 7% w/w of the tablet. Suitable disintegrants include those commonly known in the art, including clays such as bentonite, kaolin or veegum; celluloses such as microcrystalline cellulose or croscarmellose sodium; and non-ionic disintegrants such as cross-linked polyvinyl pyrrolidone sold under the trade name of crospovidone.

The lubricants may be present in an amount of about 0.1% w/w to about 2.0% w/w and in particular at from about 0.1% to about 1.5%. They may be selected from those commonly known pharmaceutically acceptable lubricants, e.g. colloidal silicon dioxide, magnesium stearate, talc and the like.

The drug and excipients may be granulated following either the wet or the dry granulation process. However, wet granulation was the more suitable of the two. The fluid uptake during granulation and the moisture content in the final granules played an important role in determining the hardness, friability and the disintegration time of the resulting tablets. The fluid uptake by the dry blend was preferably more than 10% during granulation because a fluid uptake less than 10% resulted in flow problems leading to weight variation, sticking, and picking. The maximum fluid uptake was preferably less than 16% as more than that resulted in a fluid mass and wet massing.

The granules thus prepared were dried and the effect of the moisture content in the granules in the tablet formulation was monitored. It was observed that granules containing more than approximately 3% to 4% moisture content resulted in a robust tablets.

The tablets may further be coated. The coating may be for aesthetic appeal (such as film coating) or to provide enteric or sustained release properties.

The following examples further exemplify the invention and are not intended to limit the scope of the invention.

TABLE 1
COMPOSITION OF VALACYCLOVIR
HYDROCHLORIDE TABLET

| | Quantity (mg) | | | |
|---|---|---|---|---|
| Ingredients | Example-1 | Example-2 | Example-3 | Example-4 |
| INTRAGRANULAR | | | | |
| Valacyclovir | 584.03 | 584.03 | 584.03 | 1112.55 |
| hydrochloride | | | | |
| Microcrystalline | 87.97 | 80.97 | 60.97 | 219.45 |
| cellulose | | | | |
| Crospovidone | 14 | 14 | 14 | 14 |
| Povidone K 30 | 7 | 7 | 7 | 37 |
| Povidone K 90 | — | — | — | 3 |
| Purified water | q.s. | q.s. | q.s. | q.s. |
| EXTRAGRANULAR | | | | |
| Microcrystalline | — | — | 5 | — |
| cellulose | | | | |
| HPMC E5 Premium | — | 7 | — | — |
| HPC-L | — | — | 14 | — |
| Magnesium stearate | 7 | 7 | 7 | 14 |
| TABLET WT. | 700 | 700 | 700 | 1400 |
| HARDNESS | 15-18 kP | 20-28 kP | 25-38 kP | 25-28 kP |
| RANGE | | | | |
| FRIABILITY | >1.0% | 0.2% | 0.01% | 0.2% |
| Disintegration Time | 20 | 20 | 22 | 35-38 min. |
| (Minutes) | | | | |

One process for preparing tablets of valacyclovir hydrochloride is as follows:

1. Valacyclovir hydrochloride, microcrystalline cellulose and crospovidone were sifted through a #44 BSS sieve and mixed.

2. Povidone K-30 and (K-90 in example 4) were dissolved in purified water and the bulk of step-1 was granulated using this solution

3. The granules of step 2 were dried in a fluidized bed dryer at 60° C. until a water content of 5-8% w/w was obtained.

4. Extragranular excipients were sifted through a #44 BSS sieve and blended with the granules of step 3.

5. The resulting granules of step 4 were compressed into tablets.

6. The tablets of step 5 were coated with a 12% w/w coating dispersion of Opadry in purified water until a weight build up of 3.0% w/w was obtained.

As known in the art, one suitable method for measuring the friability of a tablet is as follows. First, take ten tablets and accurately weigh the tablet sample. Then, place the tablets in the drum of a Van Kel friability apparatus (Van Kel Industries, Inc., Edison, N.J.,) rotate the drum one hundred times, and remove all the tablets. Next, remove any loose dust from the tablets, remove any broken tablets, weigh, and calculate the weight loss.

As is also known in the art, one suitable method of measuring the hardness of a tablet is as follows. Manually place the sample onto the sample support of the hardness tester and start the test. In so doing, the driven force jaw moves towards the sample. As soon as it touches the tablet it starts to increase the force as per selected force rate until the tablet is broken. The maximum force, shown as hardness in Kilopond (Kp), is provided on the display of the measurement apparatus.

A hydrated form of valacyclovir hydrochloride having a particle size of less than 250 μm and a water of hydration between 5-7% was used in the four formulations. The tablets containing extragranular binding agent showed better hardness and also low friability. Tablets prepared in accordance with Example 2 were used for all subsequent studies.

TABLE 2
EFFECT OF PARTICLE SIZE DISTRIBUTION OF VALACYCLOVIR HYDROCHLORIDE

| Valacyclovir | | | | |
|---|---|---|---|---|
| hydrochloride | #1 | #2 | #3 | #4 |
| % RETAINED ON | 4.2% | NIL | NIL | NIL |
| #22 (710 μm) | | | | |
| % RETAINED ON | 14.2% | 18.6% | NIL | NIL |
| #44 (355 μm) | | | | |
| % RETAINED ON | 24.7% | 25.7% | 23.7% | NIL |
| #60 (250 μm) | | | | |
| Hardness (kP) | 10 kP | 12 kP | 15 kP | 28 kP |
| Friability | >2% | >2% | >1% | .008% |
| (% release) | | | | |
| Disintegration | <6 | 6-8 | 10-12 | 15-20 |
| Time (minutes) | | | | |
| Coating | Tablets could | Tablets could | Tablets when | Tablets were |
| | not be coated | not be coated | coated showed | coated under |
| | | | tendency to | normal coating |
| | | | crack and edge | parameters |
| | | | chipping and | |
| | | | erosion. | |

Tablets were formulated as given in Example 2 and Example 4 of Table 1 using drug containing a valacyclovir hydrochloride particle size of less than 355 μm, or more particularly less than 250 μm had excellent hardness and low friability (# 4 above). When the valacyclovir hydrochloride particles used in the composition had more than 20% of the particles more than 250 μm and the rest below it, the granules of that batch were difficult to compress, and had low hardness and the friability was more than 1%.

TABLE 3
EFFECT OF FLUID UPTAKE

| Parameters | | | | | |
|---|---|---|---|---|---|
| studied | #1 | #2 | #3 | #4 | #5 |
| Fluid uptake | 8% | 12% | 14% | 16% | 18% |
| Core tablets | Tablets showed | Tablets took | Tablets took | Tablets took high | Tablets could |
| Parameters | capping at hardness | hardness up to | hardness up to | hardness, but processing | not be compressed. |
| (observation) | above 25 kP | 35 kP | 48 kP | required wet massing | |
| Friability | >2% | <1% | 0.002% | 0.002% | >1% |
| Disintegration | 12-14 | 15-20 | 15-20 | 15-20 | — |
| Time (minutes) | | | | | |

The following observations were made about the processing of Examples 1-5 of Table 3. In Example 1, the tablets were difficult to coat. In Examples 2 and 3, the tablets took a hardness up to 35 kP and 48 kP with ease of compressibility. There was no sticking or picking encountered. The friability improved with fluid uptake. The tablets of Examples 2 and 3 also showed no capping, lamination, cracking, or chipping during coating or stress testing. In Example 4, the wet massing was difficult. Because the granules of Example 4 were hard, the formulation became dependent on the percentage of fines and particle size distribution of the blend for compression. Since the granules are hard, they are not cohesive. The tablets of Example 5 were very friable.

As can be seen above, the fluid taken up by the dry blend during granulation played an important role in determining the hardness and friability of the resulting tablets. Fluid uptake during granulation of between 10-16% was most suited for valacyclovir hydrochloride tablets. After granulation tablets were formulated as given in Example 2 of Table 1.

TABLE 4
EFFECT OF WATER CONTENT IN THE DRIED GRANULES
PRIOR TO ADDITION OF EXTRAGRANULAR EXCIPIENTS

| Parameters studied | #1 | #2 | #3 | #4 |
|---|---|---|---|---|
| Water content of | 2.8% w/w | 5.4% w/w | 8.9% w/w | 12.3% w/w |
| dry granules | | | | |
| Friability | >1% | 0.45% | 0.005% | 0.002% |
| Hardness (range) | 20-25 kP | 25-30 kP | 25-35 kP | 25-35 kP |
| Disintegrant Time | 12-15 min | 15-20 min | 16-20 min | 16-20 min |

| | | |
|---|---|---|
| Processing | Tablets had to be coated | Tablets were coated under normal coating |
| | with pan rotating at minimum | parameters with no issues of cracking/chipping. |
| | speed and high spray rate to | |
| | prevent edge chipping. | |

The water content in the dried granules prior to their being mixed with the extragranular excipients and tabletting was measured for its effect on the hardness and friability. As can be seen from the data above granules having water content of more than 5.0% (i.e., Examples 2-4) when formulated as tablets had good hardness and low friability.

While several particular forms of the inventions have been described, it will be apparent that various modifications and combinations of the inventions detailed in the text can be made without departing from the spirit and scope of the inventions. Further, it is contemplated that any single feature or any combination of optional features of the inventive variations described herein may be specifically excluded from the claimed inventions and be so described as a negative limitation. Accordingly, it is not intended that the inventions be limited, except as by the appended claims.

We claim:

1. A tablet comprising a hydrated form of valacyclovir hydrochloride having a water of hydration content of more than approximately 3% w/w and a particle size of less than approximately 355 μm.

2. The tablet according to claim 1, wherein the valacyclovir hydrochloride has a water of hydration content of more than approximately 4% w/w.

3. The tablet according to claim 1, wherein the valacyclovir hydrochloride has a water of hydration content of between approximately 3% w/w and approximately 16% w/w.

4. The tablet according to claim 1, wherein the valacyclovir hydrochloride has a particle size of less than approximately 250 μm.

5. The tablet according to claim 1, wherein the valacyclovir hydrochloride concentration comprises at least approximately 50% w/w of the tablet.

6. The tablet according to claim 1, wherein the tablet has a friability and the friability of the tablet does not exceed approximately 1% w/w.

7. The tablet according to claim 1, wherein the tablet has a hardness and the hardness of the tablet is at least approximately 10 kP.

8. The tablet according to claim 1, further comprising one or more pharmaceutically acceptable excipients.

9. The tablet according to claim 8, wherein the pharmaceutically acceptable excipients comprise one or more of a filler, binding agent, disintegrant and lubricant.

10. The tablet according to claim 9, wherein the filler comprises one or more of dicalcium phosphate and microcrystalline cellulose.

11. The tablet according to claim 9, wherein filler comprises from about 5% to about 40% w/w of the tablet.

12. The tablet according to claim 9, wherein the binding agent comprises one or more of hydroxypropyl methylcellulose, hydroxypropyl cellulose, and polyvinyl pyrrolidone.

13. The tablet according to claim 9, wherein the binding agent comprises between 0.05% and 5% w/w of the tablet.

14. The tablet according to claim 13, wherein a portion of the binding agent is present extra granularly as a dry binding agent.

15. The tablet according to claim 14, wherein the extra granular dry binding agent comprises between approximately 0.05% and approximately 2% w/w of the tablet.

16. The tablet according to claim 9, wherein the disintegrant comprises one or more of clays, kaolin, bentonite, veegum; celluloses, microcrystalline cellulose, croscarmellose sodium, non-ionic disintegrants, and crospovidone.

17. The tablet according to claim 9, wherein the disintegrant comprises from approximately 0.5% to approximately 7% w/w of the tablet.

18. The tablet according to claim 9, further comprising a film coating.

19. A tablet comprising:
an intragranular portion comprising at least approximately 50% w/w of a hydrated form of valacyclovir hydrochloride having a water of hydration content of more than approximately 3% w/w and a particle size less than approximately 355 μm, at least one filler, at least one binding agent, and at least one disintegrant; and
an extragranular portion comprising at least one lubricant, wherein the friability of the tablet does not exceed approximately 1% and the hardness is at least approximately 10 kP.

20. A tablet comprising:
an intragranular portion comprising at least approximately 50% w/w of a hydrated form of valacyclovir hydrochloride having a water of hydration content of more than approximately 3% w/w and particle size less than approximately 355 μm, at least one filler, at least one binding agent, and at least one disintegrant present within the granules of the tablet; and
an extragranular portion comprising at least one lubricant and at least one binding agent, wherein the friability of the tablet does not exceed approximately 1%, the hardness is at least approximately 10 kP.

21. The tablet according to claim 20, wherein the binding agent in the intragranular portion and the binding agent in the extragranular portion are of the same material composition.

22. A method of treatment of a viral infection in a mammal comprising administering to the mammal one or more tablets to administer an effective anti-viral amount of valacyclovir hydrochloride, the tablet comprising a hydrated form of valacyclovir hydrochloride having a water of hydration content of more than approximately 3% w/w and a particle size of less than approximately 355 μm

23. The method of treatment of claim 22, wherein the virus comprises a DNA virus.

24. The method of treatment of claim 22, wherein the virus comprises one or more of herpes simplex 1, herpes simplex 2, varicella zoster, cytomegalovirus, Epstein-Barr viruses, human herpes virus-6 (HIV-6), and hepatitis B virus.

25. The method of claim 22, wherein the virus comprises one or more of a papilloma or wart virus.

26. The method of claim 22, further comprising administering the tablet with a second active compound.

27. The method of claim 26, wherein the second active compound comprises zidovudine.

28. A process for preparing a tablet comprising at least approximately 50% w/w of a hydrated form of valacyclovir hydrochloride having a water of hydration content of more than approximately 3% w/w and a particle size less than approximately 355 μm and one or more of at least one binding agent, at least one filler, at least one disintegrant and at least one lubricant, the process comprising:
forming granules of valacyclovir hydrochloride; and
blending an optional portion of the at least one binding agent and a lubricant with the granules,
wherein the hardness of the tablet is at least approximately 10 kP and the friability is not more than approximately 1%.

29. The process according to claim 28, wherein forming the granules comprises:
mixing the valacyclovir hydrochloride and the one or more of the at least one binding agent, the at least one filler, and the at least one disintegrant;
granulating with a granulating solution to form granules;
drying the granules;
blending the granules with a lubricant; and
compressing the blended mixture to form a tablet.

30. The process according to claim 29, wherein blending the granules with a lubricant further comprises blending with a binding agent.

31. The process according to claim 28, wherein forming the granules comprises:
dissolving the binding agent in a granulating solution;
adding and mixing to the granulating solution the valacyclovir hydrochloride and the one or more of the at least one binding agent, the at least one filler, and the at least one disintegrant;
granulating with a granulating solution to form granules;
drying the granules;
blending the granules with a lubricant; and
compressing the blended mixture to form a tablet.

32. The process according to claim 31, wherein blending the granules with a lubricant further comprises blending with a binding agent.

33. The process according to claim 28, wherein the granulation results in a fluid uptake of between 8-16%.

34. The process according to claim 29, wherein the fluid uptake after granulation comprise between approximately 12 and approximately 16%.

35. The process according to claim 29, wherein the granules are dried to a moisture content of more than approximately 4% w/w.

36. The process according to claim 29 wherein the extra granular binding agent is first blended with the lubricant before blending with the granules.

37. The process according to claim 29 wherein the extra granular binding agent is added separately from the lubricant.

38. A method of improving one or both of friability and hardness of a tablet comprising valacyclovir hydrochloride, the method comprising:
reducing the particle size of a hydrated form of valacyclovir hydrochloride, the valacyclovir hydrochloride having a water of hydration content of more than approximately 3% w/w.

39. The method of improving one or both of friability and hardness of claim 38, wherein the particle size is less than approximately 355 μm.

40. A method of improving one or both of friability and hardness of a tablet comprising valacyclovir hydrochloride having a particle size of less than approximately 355 μm, the method comprising:
forming the tablet from a hydrated form of valacyclovir hydrochloride having a water of hydration content of more than approximately 3% w/w.

41. A tablet comprising a hydrated form of valacyclovir hydrochloride characterized by the absence of colloidal silicon dioxide and extra granular microcrystalline cellulose.

42. The tablet of claim 41, wherein the valacyclovir hydrochloride has a water of hydration content of more than approximately 3% w/w and a particle size of less than approximately 355 μm.

43. The tablet of claim 1, which is further free or substantially free of both silicon dioxide and microcrystalline cellulose.
